(19) United States
(12) Patent Application Publication
(10) Pub. No.: US 2004/0024378 A1
(43) Pub. Date: Feb. 5, 2004

(54) Adhesive tape

(76) Inventor: Chen-Cheng Huang, Taipei (TW)

Correspondence Address: FOLEY AND LARDNER, SUITE 500, 3000 K STREET NW, WASHINGTON, DC, 20007, US

(21) Appl. No.: 10/208,788

(22) Filed: Aug. 1, 2002

(51) Int. Cl. A61F 13/15

(52) U.S. Cl. 604/389

(57) ABSTRACT

An adhesive tape includes a backing substrate having opposite first and second surfaces and formed with a plurality of through-holes, each of which is confined by a hole-confining wall. The backing substrate is formed with a plurality of annular flanges, each of which extends from the hole-confining wall of a respective one of the through-holes at the second surface. An adhesive layer is coated on the second surface.

### BACKGROUND OF THE INVENTION

1. Field of the Invention

This invention relates to an adhesive tape, more particularly to an adhesive tape with a plurality of annular flanges projecting therefrom.

2. Description of the Related Art

FIGS. 1 and 2 illustrate a conventional diaper 10 that includes a back sheet 14 with front and rear belt parts 15, 16, and a pair of conventional fastener tapes 11 secured to two opposite ends of the rear belt part 16 for attaching to two opposite ends of the front belt part 15, respectively. Each of the fastener tapes 11 includes a backing substrate 12 and an adhesive layer 13 coated on the backing substrate 12. The conventional fastener tapes 11 is disadvantageous in that since the fastener tapes 11 are required to be firmly bonded to the front belt part 15 when the diaper 10 is in use, the adhesive layer 13 is normally made from an adhesive that has a relatively strong adhesion, which makes the fastener tapes 11 difficult to peel from the front belt part 15 for removal by the user, and which tends to damage the front belt part 15.

### SUMMARY OF THE INVENTION

Therefore, the object of the present invention is to provide an adhesive tape that is capable of overcoming the aforesaid drawbacks of the prior art.

According to the present invention, an adhesive tape comprises: a backing substrate having opposite first and second surfaces, and formed with a plurality of through-holes, each of which extends through the first and second surfaces and each of which is confined by a hole-confining wall, the backing substrate being further formed with a plurality of annular flanges, each of which extends outwardly from the hole-confining wall of a respective one of the through-holes at the second surface, the annular flanges being plastically deformable; and an adhesive layer coated on the second surface.

### BRIEF DESCRIPTION OF THE DRAWINGS

In drawings which illustrate embodiments of the invention,

FIG. 1 is a perspective view of a conventional diaper;

FIG. 2 is a fragmentary sectional view of a conventional fastener tape of the diaper of FIG. 1;

FIG. 3 is a fragmentary perspective view of a first embodiment of an adhesive tape of this invention;

FIG. 4 is a fragmentary sectional view of the adhesive tape of FIG. 3;

FIG. 5 is a fragmentary sectional view of a second preferred embodiment of the adhesive tape of this invention;

FIG. 6 is a fragmentary sectional view of a third preferred embodiment of the adhesive tape of this invention;

FIG. 7 is a perspective view of a diaper with the adhesive tape of this invention; and

FIG. 8 is a perspective view of a box with the adhesive tape of this invention.

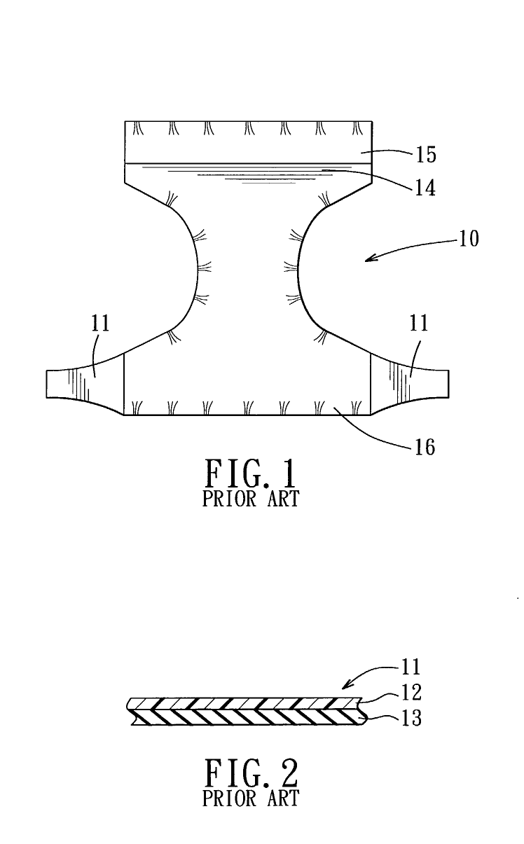

### DETAILED DESCRIPTION OF THE PREFERRED EMBODIMENTS

For the sake of clarity, like elements are denoted by the same reference numerals throughout the disclosure.

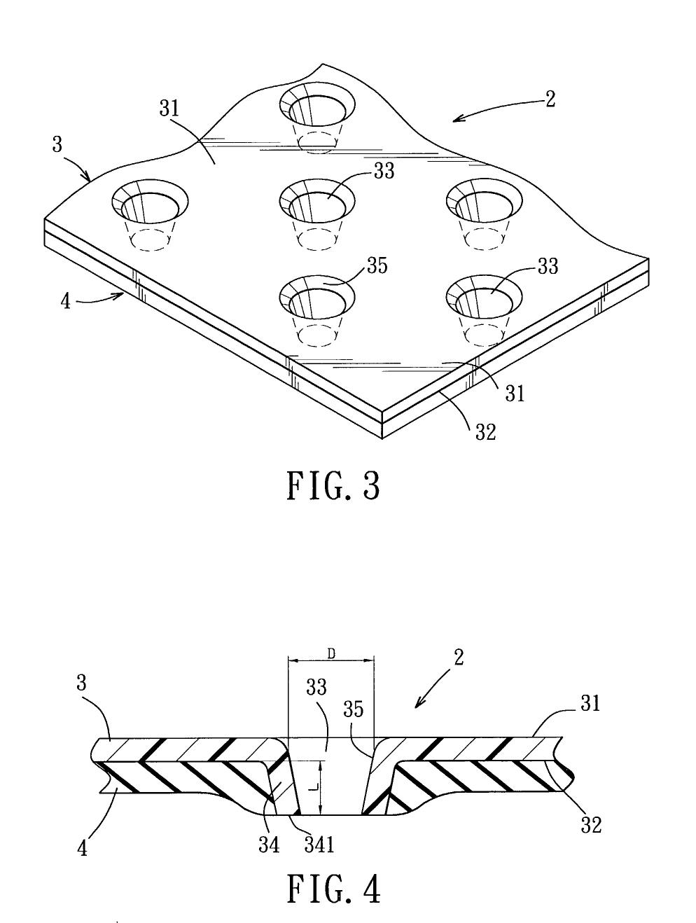

FIGS. 3 and 4 illustrate a first preferred embodiment of an adhesive tape 2 of this invention.

The adhesive tape 2 includes: a backing substrate 3 having opposite first and second surfaces 31, 32, and formed with a plurality of through-holes 33, each of which extends through the first and second surfaces 31, 32 and each of which is confined by a hole-confining wall 35, the backing substrate 3 being further formed with a plurality of annular flanges 34, each of which extends outwardly from the hole-confining wall 35 of a respective one of the through-holes 33 at the second surface 32; and a first adhesive layer 4 coated on the second surface 32. The annular flanges 34 are plastically deformed when the first adhesive layer 4 of the adhesive tape 2 is bonded to a supporting surface (not shown), which results in an urging force against the supporting surface, and which, in turn, facilitates peeling of the adhesive tape 2 from the supporting surface.

The backing substrate 3 is preferably made from a plastic material. The first adhesive layer 4 is preferably made from a pressure sensitive adhesive so as to permit the adhesive tape 2 to be peelable. Each of the annular flanges 34 is tapered, is reduced from the hole-confining wall 35 of the respective one of the through-holes 33, and is formed along with the respective through-hole 33 by breaking the backing substrate 3 with a tool (not shown).

Each of the through-holes 33 has a diameter (D) Each of the annular flanges 34 has a free end 341 that is vertically spaced apart from the second surface 32 by a distance (L) such that the ratio of the distance (L) between the free end 341 and the second surface 32 to the diameter (D) of a respective one of the through-holes 33 is greater than 0.1.

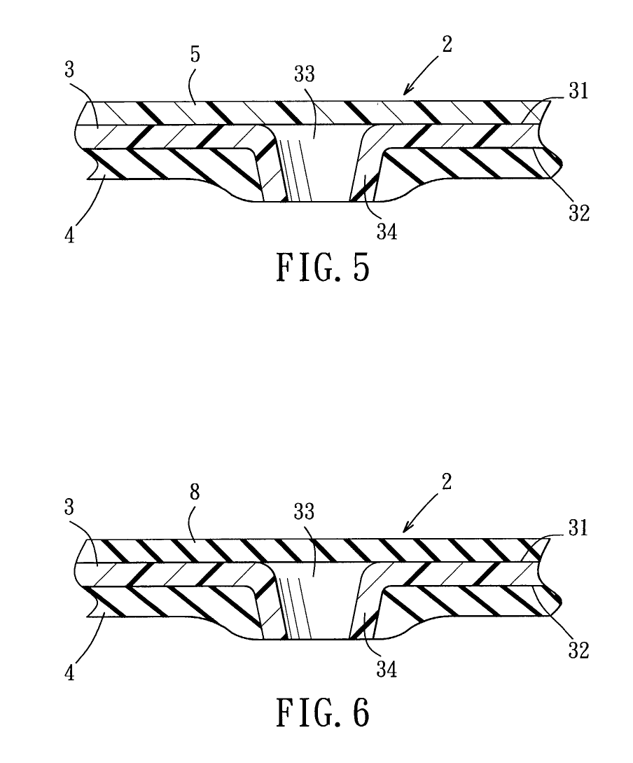

FIG. 5 illustrates a second preferred embodiment of the adhesive tape 2 of this invention. The adhesive tape 2 of this embodiment is similar to that shown in FIG. 4, except that a reinforcing substrate 5 is superimposed on and is bonded to the first surface 31 of the backing substrate 3 so as to increase mechanical strength of the adhesive tape 2.

FIG. 6 illustrates a third preferred embodiment of the adhesive tape 2 of this invention. The adhesive tape 2 of this embodiment is similar to that shown in FIG. 4, except that a second adhesive layer 8 is coated on the first surface 31 of the backing substrate 3.

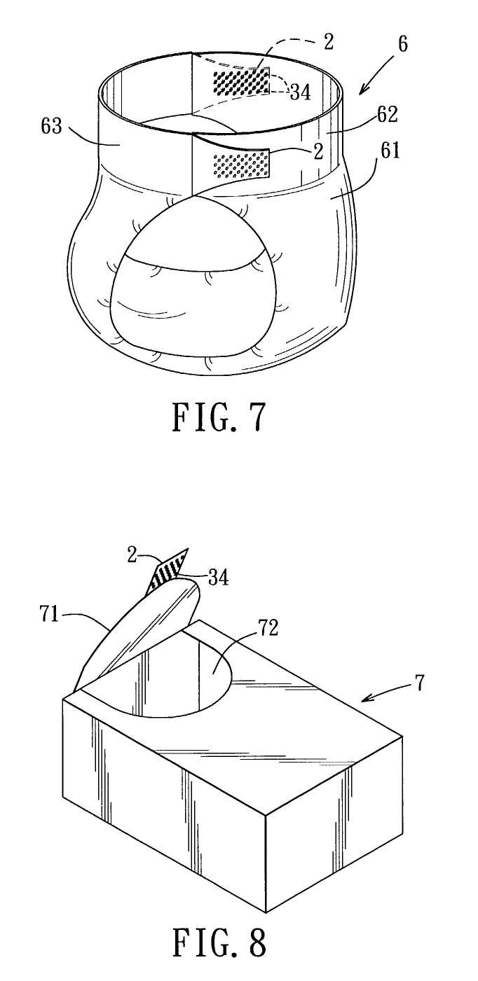

Referring now to FIG. 7, in combination with FIGS. 3 and 4, the adhesive tape 2 of this invention can be used as a fastener tape for a diaper 6. The diaper 6 includes a back sheet 61 with front and rear belt parts 62, 63, each of which has two opposite ends. A pair of the adhesive tapes 2 are secured to the opposite ends of the rear belt part 63 for releasably attaching to the opposite ends of the front belt part 61.

Referring now to FIG. 8, in combination with FIGS. 3 and 4, the adhesive tape 2 of this invention can also be used for fastening a cover flap 71 to a box 7 in order to cover an opening 72 in the box 7.

With the design of the annular flanges 34 in the adhesive tape 2 of this invention, the aforesaid drawbacks as encountered in the prior art can be eliminated.

With the invention thus explained, it is apparent that various modifications and variations can be made without departing from the spirit of the present invention. It is therefore intended that the invention be limited only as recited in the appended claims.

I claim:

1. An adhesive tape comprising:
a backing substrate having opposite first and second surfaces, and formed with a plurality of through-holes, each of which extends through said first and second surfaces and each of which is confined by a hole-confining wall, said backing substrate being further formed with a plurality of annular flanges, each of which extends outwardly from said hole-confining wall of a respective one of said through-holes at said second surface, said annular flanges being plastically deformable; and
a first adhesive layer coated on said second surface.

2. The adhesive tape of claim 1, wherein each of said annular flanges is tapered and is reduced from said hole-confining wall of the respective one of said through-holes.

3. The adhesive tape of claim 2, wherein each of said through-holes has a diameter, each of said annular flanges having a free end that is vertically spaced apart from said second surface by a distance such that the ratio of the distance between said free end and said second surface to the diameter of a respective one of said through-holes is greater than 0.1.

4. The adhesive tape of claim 1, wherein said first adhesive layer is made from a pressure sensitive adhesive.

5. The adhesive tape of claim 1, further comprising a reinforcing substrate superimposed on and bonded to said first surface of said backing substrate so as to increase mechanical strength of said adhesive tape.

6. The adhesive tape of claim 1, further comprising a second adhesive layer coated on said first surface of said backing substrate.

7. A diaper comprising:
a back sheet with front and rear belt parts, each of which has two opposite ends; and
a pair of adhesive tapes secured to said opposite ends of said rear belt part for releasbly attaching to said opposite ends of said front belt part, respectively, each of said adhesive tapes including:
a backing substrate having opposite first and second surfaces, and formed with a plurality of through-holes, each of which extends through said first and second surfaces and each of which is confined by a hole-confining wall, said backing substrate being further formed with a plurality of annular flanges, each of which extends outwardly from said hole-confining wall of a respective one of said through-holes at said second surface, said annular flanges being plastically deformable, and
an adhesive layer coated on said second surface.
